(19) United States
(12) Patent Application Publication
(10) Pub. No.: US 2008/0057105 A1
(43) Pub. Date: Mar. 6, 2008

(54) MEDICAL DEVICES HAVING NANOSTRUCTURED COATING FOR MACROMOLECULE DELIVERY

(75) Inventors: Liliana Atanasoska, Edina, MN (US); Jan Weber, Maastricht (NL); Robert W. Warner, Woodbury, MN (US); Michele Zoromski, Minneapolis, MN (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 11/847,934

(22) Filed: Aug. 30, 2007

Related U.S. Application Data
(60) Provisional application No. 60/842,383, filed on Sep. 6, 2006.

(51) Int. Cl. A61F 2/02; A61K 31/7052; A61P 43/00

(52) U.S. Cl. 424/426; 514/44

(57) ABSTRACT

A medical device having a biodegradable coating comprising an inorganic material complexed to macromolecules. Biodegradation of the biodegradable coating releases nanoparticles of the inorganic material with macromolecules complexed to the released nanoparticles. The inorganic material may be applied directly onto the medical device as a nanostructured coating or be dispersed within or under a layer of biodegradable polymer. The medical device body may comprise a biodegradable metallic material. Also provided is a method of delivering macromolecules to body tissue using the medical device of the present invention.

### RELATED APPLICATIONS

This application claim benefit of 60/842,383, filed Sep. 6, 2006, which is incorporated herein in its entirety

### TECHNICAL FIELD

The present invention relates to coated medical devices. More specifically, the present invention relates to medical devices having a nanostructured coating for carrying and releasing macromolecules.

### BACKGROUND

Many implantable medical devices have a drug-loaded coating designed to improve the effectiveness of the medical device. For example, some coronary artery stents are coated with a drug which is eluted from the stent to prevent some of the unwanted effects and complications of implanting the stent. Some have also attempted to use medical device coatings as a means to provide gene therapy. For example, some investigators have used stents with a coating that elutes naked DNA encoding human vascular endothelial growth factor (VEGF-2) to treat cells in the arterial wall. Naked DNA, however, is not an efficient means for transfecting cells. See Schmidt-Wolf et al., Trends in Molecular Medicine 9(2):67-72 (2003), which is incorporated by reference herein. Thus, there is a need for a medical device that delivers macromolecules, such as DNA, more effectively to tissue cells.

### SUMMARY OF THE INVENTION

The present invention is directed to a medical device that provides a means of delivering macromolecules. In an embodiment, the present invention provides a medical device comprising a medical device body, such as a stent; a biodegradable coating comprising an inorganic material disposed on the medical device body; and macromolecules conjugated to the inorganic material; wherein biodegradation of the coating releases nanoparticles of the inorganic material, and wherein the macromolecules are conjugated to the released nanoparticles. In an embodiment, the inorganic material forms a nanostructured layer. The inorganic materials may comprise metal salts, metal oxides, or metal hydroxides. The macromolecules may be conjugated to the exterior or interior of the nanoparticles by ionic bonding. The macromolecules may be polynucleotides. The nanoparticles may be released individually or in aggregates. The biodegradable coating may further comprise a buffering agent.

In another embodiment of the present invention, the biodegradable coating further comprises a biodegradable polymer. In yet another embodiment, the medical device body (e.g., a stent) comprises a biodegradable metallic material, and the inorganic material comprises metal phosphates. Biodegradation of the metallic material can release metal ions and biodegradation of the coating can release phosphate ions such that the metal ions and phosphate ions combine to form metal phosphate nanoparticles, and wherein macromolecules are conjugated to the metal phosphate nanoparticles. Biodegradation of the metallic material can involve a corrosive process and the coating may modulate the corrosive process. The coating and the medical device body can form a galvanic couple.

The present invention also provides a method of delivering macromolecules to body tissue comprising the steps of providing a medical device of the present invention and implanting the medical device in a subject's body.

### BRIEF DESCRIPTION OF THE DRAWINGS

FIG. 1 is a high magnification view of an exemplary nanostructured coating.

FIG. 2 show nanoparticles according to an embodiment of the present invention and a schematic representation of the transfection mechanism.

FIG. 3 shows an aggregate of nanoparticles according to an alternate embodiment of the present invention.

### DETAILED DESCRIPTION

The present invention provides a medical device having a biodegradable coating comprising an inorganic material complexed to macromolecules. Biodegradation of the biodegradable coating releases nanoparticles of the inorganic material with macromolecules complexed to the released nanoparticles.

In an embodiment of the present invention, the inorganic material is applied directly onto the medical device as a nanostructured coating. Nanostructures of the present invention include structures having at least one characteristic domain with a dimension in the nanometer range, such as 500 nm or less. The domain dimension may be along the largest or smallest axis of the structure. The domains may be any physical feature or element of the nanostructure, such as pores, matrices, particles, or grains. Biodegradability of any material of the present invention includes the process of breaking down or degrading by either chemical, including corrosive, or physical processes upon interaction with a physiological environment. The products of the degradation process may be soluble, such as metal cations, or insoluble precipitates. Insoluble precipitates may form particles, such as metal phosphate nanoparticles.

The inorganic material is biocompatible and may be a metal salt, metal oxide, or metal hydroxide. The metal may be a metal in which its cation forms ionic complexes with DNA, such as Ca²⁺, Mg²⁺, Mn²⁺, or Ba²⁺. The inorganic material may also be an inorganic phosphate or a metal phosphate such as magnesium phosphate, manganese phosphate, barium phosphate, calcium phosphate, or mixtures or combinations of these, such as calcium-magnesium phosphate.

The inorganic material is applied to the medical device by any known method of deposition that forms a nanostructured coating. These methods can include sol-gel, layer-by-layer (LbL) coating, self-assembly, chemical or physical vapor deposition, or spraying. The nanostructured coating can also be formed by the method described in Kouisni et al., Surface Coating & Technology 192:239-246 (2005), which is incorporated by reference herein. Kouisni describes creating a zinc phosphate coating on magnesium alloy AM60 (containing 6% Al and 0.28% Mn) by immersing the alloy in a 3.0 pH phosphating bath containing phosphoric acid, phosphate ions, nitrates, nitrites, zinc, and fluorides.

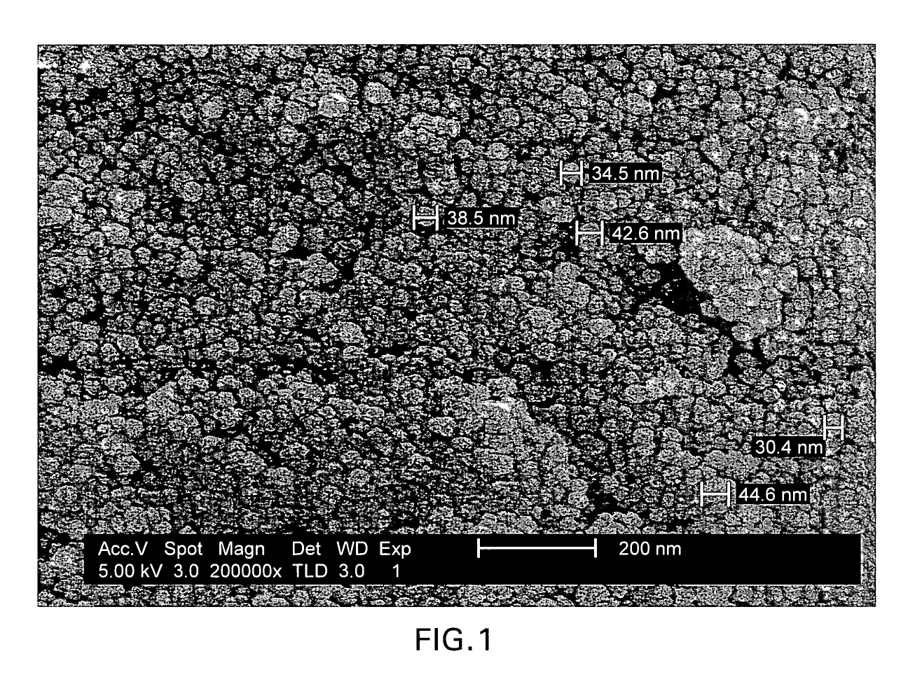

FIG. 1 shows a high magnification view of an exemplary nanostructured coating (image obtained from Sol-Gel Technologies) that can be created by sol-gel techniques for use with the present invention. In this particular example, the characteristics domains of the nanostructure are nanoparticles which range in size from about 30 to about 45 nm in diameter. This example is provided merely to illustrate and is not intended to be limiting.

Macromolecules are conjugated to the inorganic material by ionic bonding. The macromolecules can include, for example, polynucleotides, peptides, proteins, enzymes, polyamines, polyamine acids, polysaccharides, lipids, as well as small molecule compounds such as pharmaceuticals. The polynucleotides may be DNA or RNA, which can encode a variety of proteins or polypeptides, and the polynucleotides may be inserted into recombinant vectors such as plasmids, cosmids, phagemids, phage, viruses, and the like. There is no limit to the size of the polynucleotides, as described in Schmidt-Wolf et al., Trends in Molecular Medicine 9(2):67-72 (2003), which is incorporated by reference herein. The macromolecules may be attached to the external surface of the nanostructure domains, incorporated or dispersed within the nanostructure domains, or within the matrix of the nanostructure.

After the medical device is implanted in the subject's body and exposed to a physiologic environment, the nanostructured coating undergoes biodegradation. Biodegradation of the nanostructured coating may be a physical process, such as the frictional and mechanical forces created by the flow of fluid or blood. The biodegradation may also be a chemical process, such as corrosion or hydrolysis.

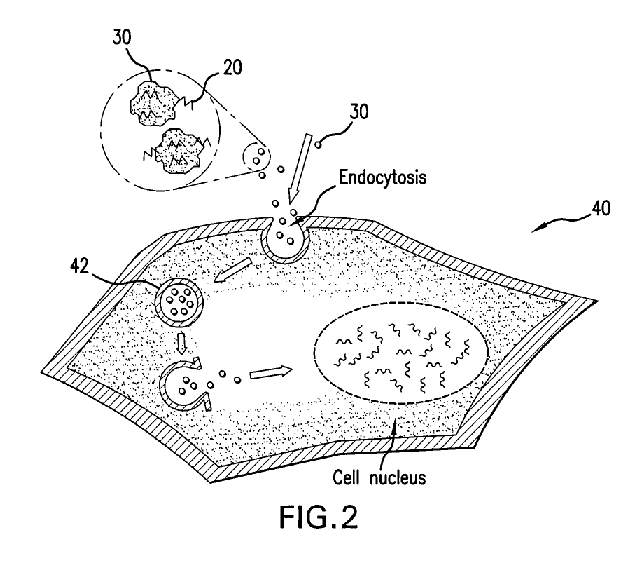

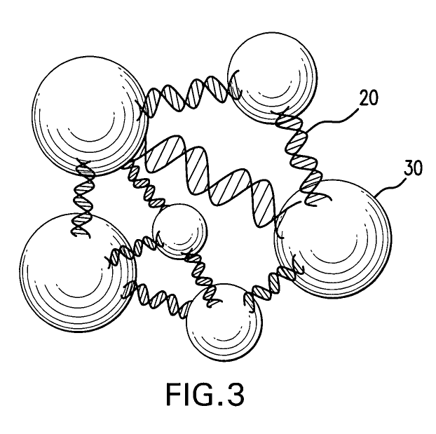

Referring to FIG. 2, biodegradation of the nanostructured coating results in the release of nanoparticles 30 of the inorganic material into the surrounding fluid or tissue. In an embodiment, macromolecules 20 are conjugated to the surface of nanoparticles 30. In an alternate embodiment, macromolecules 20 are incorporated or dispersed within nanoparticle 30, or encapsulated within nanoparticle 30, as described in Bhakta et al., Biomaterials 26:2157-2163 (2005), which is incorporated by reference herein. The nanoparticles may be released individually or in aggregates, as shown in FIG. 3, such that the aggregates themselves are nanoparticles. The nanoparticles are of sizes that allow them to serve as polynucleotide vectors in cell transfection. For example, inorganic calcium-magnesium phosphate nanoparticles of up to 500 nm have been shown to be effective in gene transfection of Hela and NIH-3T3 cells, as described in Chowdhury et al., Gene 341:77-82 (2004), which is incorporated by reference herein.

The present invention provides a medical device coated with DNA-loaded nanoparticles that can be more effective in DNA transfection than naked DNA. In particular, nanoparticles of calcium phosphate, calcium-magnesium phosphate, manganese phosphate, and magnesium phosphate have been demonstrated to be effective vectors for plasmid DNA transfection into cells, as described in Bhakta et al., Biomaterials 26:2157-63 (2005); Chowdhury et al., Gene 341:77-82 (2004); and U.S. Pat. No. 6,555,376 (Maitra et al.), all of which are incorporated by reference herein. Referring again to FIG. 2 and without being bound by theory, it is believed that DNA-loaded nanoparticles 30 enter a cell 40 through the process of endocytosis. Inside the cell 40, the nanoparticles 30 are stored in endosomes 42 wherein the mildly acidic pH causes the DNA to be released from the nanoparticles.

One example of a medical device that can be coated with the nanostructured inorganic material of the present invention is a stent. Plasmid DNA encoding for genes that can be used to treat vascular diseases and conditions, such as the gene for human vascular endothelial growth factor-2 (VEGF-2), can be conjugated to the inorganic material. DNA-carrying nanoparticles released from the coating can be taken up by cells in the vascular wall through endocytosis or any other transfection mechanism.

In another embodiment of the present invention, the body of the medical device is formed of a biodegradable metallic material, such as the metal alloys used in the biodegradable coronary stents described in U.S. Pat. No. 6,287,332 (Bolz et al.), which is incorporated by reference herein. In these embodiments, the body of the implanted medical device will biodegrade into harmless constituents inside the subject's body. The biodegradation may involve a corrosive process.

In this embodiment, a nanostructured coating comprising a metal phosphate material is disposed on the medical device body and macromolecules are conjugated to the metal phosphate material. As in previous embodiments, biodegradation of the nanostructured coating results in the release of nanoparticles, wherein macromolecules are conjugated to the nanoparticles. In this embodiment, nanoparticles can also be formed by the recombination of metal ions resulting from the biodegradation of the medical device body and phosphate ions resulting from the biodegradation of the metal phosphate coating. The metal ions combined with phosphate ions can precipitate into nanoparticles wherein macromolecules are conjugated to the nanoparticles, as described in Haberland et al., Biochimica et Biophysica Act 1445:21-30 (1999), which is incorporated by reference herein.

Phosphate coatings on metal substrates are known to slow the corrosion of the underlying metal. Examples of such phosphate coatings include coatings formed of zinc phosphate, manganese phosphate, calcium phosphate, and iron phosphate, as described in Weng et al., Surface Coating & Technology 88:147-156 (1996), which is incorporated by reference herein. Thus, in this embodiment, the metal phosphate coating can be used to alter the corrosion rate of the underlying medical device body, in addition to serving as a delivery system for macromolecules.

The corrosion rate of the medical device body will vary with the composition, thickness, porosity, electrochemical properties, and mechanical properties of the inorganic phosphate coating. Therefore, one of skill in the art can adjust such factors to achieve the desired corrosion rate in the medical device body. For example, it may be desirable to slow the corrosion rate where an extended period of mechanical stability is required for effective functioning of the medical device, such as a stent supporting a vascular wall. It may also be desirable to slow the corrosion rate to reduce the amount of harmful gases, insoluble precipitates, or other by-products generated by the corrosion process. In other cases, it may be desirable to accelerate the corrosion process.

Where the coating and the medical device are formed of different metals, the two components may also form a galvanic couple, wherein electrical current is generated between the coating and medical device body with the surrounding fluid or tissue serving as the electrolyte. For example, a galvanic current may be generated between a coating formed of zinc and zinc phosphate and a medical device formed of magnesium. The galvanic current will alter the corrosion rate of the metal components of the coating or medical device. Furthermore, it is known that the application of electrical current to cells can improve DNA transfection, as described in Schmidt-Wolf et al., Trends in Molecular Medicine 9(2):67-72 (2003), which is incorporated by reference herein. Thus, the current generated by the galvanic coupling of the coating and medical device body may also be used to enhance DNA transfection.

In another embodiment of the present invention, the biodegradable coating further comprises a layer of biodegradable polymer, wherein the inorganic material with macromolecules complexed thereto is dispersed within or under the layer of biodegradable polymer. Upon implantation of the medical device, the biodegradable polymer layer is degraded by exposure to a physiologic environment, releasing the inorganic material and macromolecules.

In certain embodiments, the biodegradable coating may further comprise an electrically conductive polymer such as phosphate-doped polypyrrole. The electrically conductive polymer can form a galvanic couple with a substrate metallic medical device, and thereby control the corrosion rate of the medical device.

In certain embodiments, the coating may further comprise a buffering agent which would serve to control the pH of the local environment surrounding the medical device. For example, formation of buffer coatings on medical devices using ion-exchange resins is described in U.S. Pat. No. 5,941,843 (Atanasoska et al.), which is incorporated by reference herein. The buffering agent may be used to reduce the pH within or adjacent to the coating to increase the dissolution of the inorganic material. See Bhakta et al., Biomaterials 26:2157-63 (2005), which is incorporated by reference herein.

The medical device of the present invention is not limited to the coronary stents in the disclosed embodiments. Non-limiting examples of other medical devices that can be used with the nanostructured coating of the present invention include catheters, guide wires, balloons, filters (e.g., vena cava filters), stents, stent grafts, vascular grafts, intraluminal paving systems, pacemakers, electrodes, leads, defibrillators, joint and bone implants, spinal implants, vascular access ports, intra-aortic balloon pumps, heart valves, sutures, artificial hearts, neurological stimulators, cochlear implants, retinal implants, and other devices that can be used in connection with therapeutic coatings. Such medical devices are implanted or otherwise used in body structures or cavities such as the vasculature, gastrointestinal tract, abdomen, peritoneum, airways, esophagus, trachea, colon, rectum, biliary tract, urinary tract, prostate, brain, spine, lung, liver, heart, skeletal muscle, kidney, bladder, intestines, stomach, pancreas, ovary, uterus, cartilage, eye, bone, and the like.

The foregoing description and examples have been set forth merely to illustrate the invention and are not intended to be limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A medical device, comprising:
(a) a medical device body;
(b) a biodegradable coating comprising an inorganic material disposed on the medical device body; and
(c) macromolecules conjugated to the inorganic material;
wherein biodegradation of the coating releases nanoparticles of the inorganic material, and wherein the macromolecules are conjugated to the released nanoparticles.

2. The medical device of claim 1, wherein the inorganic material forms a nanostructured layer.

3. The medical device of claim 1, wherein the inorganic material comprises a metal salt, a metal oxide, or a metal hydroxide.

4. The medical device of claim 3, wherein the metal salt is selected from the group consisting of magnesium phosphate, calcium phosphate, calcium-magnesium phosphate, zinc phosphate, iron phosphate, barium phosphate, and manganese phosphate.

5. The medical device of claim 1, wherein the macromolecules are conjugated to the exterior of the nanoparticles.

6. The medical device of claim 1, wherein the macromolecules are conjugated to the interior of the nanoparticles.

7. The medical device of claim 1, wherein the nanoparticles are released in aggregates.

8. The medical device of claim 1, wherein the macromolecules are polynucleotides.

9. The medical device of claim 8, wherein the polynucleotides comprise a gene encoding for human vascular endothelial growth factor-2.

10. The medical device of claim 1, wherein the biodegradable coating further comprises a biodegradable polymer.

11. The medical device of claim 10, wherein the biodegradable coating further comprises an electrically conductive polymer.

12. The medical device of claim 1, wherein the biodegradable coating further comprises a buffering agent.

13. The medical device of claim 1, wherein the medical device body comprises a biodegradable metallic material.

14. The medical device of claim 13, wherein metal ions are released by biodegradation of the metallic material.

15. The medical device of claim 14, wherein phosphate ions are released by biodegradation of the coating.

16. The medical device of claim 15, wherein the metal ions and phosphate ions combine to form metal phosphate nanoparticles, and wherein the macromolecules are conjugated to the metal phosphate nanoparticles.

17. The medical device of claim 13, wherein biodegradation of the metallic material of the medical device body includes a corrosive process.

18. The medical device of claim 17, wherein the coating modulates the corrosion of the metallic material of the medical device body.

19. A method of delivering macromolecules to body tissue, comprising:
(i) providing a medical device, wherein the medical device comprises:
(a) a medical device body;
(b) a biodegradable coating comprising an inorganic material disposed on the medical device body; and
(c) macromolecules conjugated to the inorganic material;
wherein biodegradation of the coating releases nanoparticles of the inorganic material, and wherein the macromolecules are conjugated to the released nanoparticles; and
(ii) implanting the medical device in a subject's body.

20. The method of claim 19, wherein the macromolecules are polynucleotides.

21. The method of claim 20, wherein the polynucleotides comprise a gene encoding for human vascular endothelial growth factor-2.
